(19) United States
(12) Patent Application Publication
(10) Pub. No.: US 2024/0102025 A1
(43) Pub. Date: Mar. 28, 2024

(54) GENE COMBINATION FOR EXPRESSING AND PRODUCING TERREQUINONE A IN ESCHERICHIA COLI AND USE THEREOF

(71) Applicant: Shanghai Academy of Agricultural Sciences, Shanghai (CN)

(72) Inventors: Yongsheng TIAN, Shanghai (CN); Lijuan WANG, Shanghai (CN); Yongdong DENG, Shanghai (CN); Quanhong YAO, Shanghai (CN); Rihe PENG, Shanghai (CN); Jianjie GAO, Shanghai (CN); Zhenjun LI, Shanghai (CN); Wenhui ZHANG, Shanghai (CN); Bo WANG, Shanghai (CN); Jing XU, Shanghai (CN); Yu WANG, Shanghai (CN); Xiaoyan FU, Shanghai (CN); Hongjuan HAN, Shanghai (CN)

(21) Appl. No.: 18/322,932

(22) Filed: May 24, 2023

(30) Foreign Application Priority Data
Sep. 20, 2022 (CN) 202211144379.4

(51) Int. Cl. C12N 15/70; C12P 17/16

(52) U.S. Cl. CPC C12N 15/70; C12P 17/165

(57) ABSTRACT

The present disclosure provides a gene combination for expressing and producing terrequinone A in Escherichia coli and use thereof. The gene combination includes a tdiAS gene, a tdiBS gene, a tdiCS gene, a tdiDS gene, a tdiES gene, an sfpS gene, an ScCKS gene, and an AtIPKS gene with nucleotide sequences set forth in SEQ ID NOS:1 to 8. In the present disclosure, a recombinant engineered strain capable of producing terrequinone A having anti-cancer activity is obtained by separately constructing recombinant plasmids pC02 and pU03 through the eight genes and transforming the two recombinant plasmids into E. coli. The content of terrequinone A in a fermentation broth thereof is 106.3 mg/L, which has potential application value in the biopharmaceutical field.

### CROSS REFERENCE TO RELATED APPLICATION

This patent application claims the benefit and priority of Chinese Patent Application No. 202211144379.4 filed with the China National Intellectual Property Administration on Sep. 20, 2022, the disclosure of which is incorporated by reference herein in its entirety as part of the present application.

### REFERENCE TO SEQUENCE LISTING

A computer readable XML file entitled “HLP20230201303”, that was created on Apr. 6, 2023, with a file size of about 56,389 bytes, contains the sequence listing for this application, has been filed with this application, and is hereby incorporated by reference in its entirety.

### TECHNICAL FIELD

The present disclosure belongs to the field of gene engineering, in particular to a gene combination for expressing and producing terrequinone A in Escherichia coli and use thereof.

### BACKGROUND

Bis-indolequinone compounds are a group of fungal natural products with bioactivities such as antiretroviral, antidiabetic or cytotoxic bioactivities. Since the discovery of cochliodinol, the family members of bis-indolequinone compounds are expanding. Herein, terrequinone A, which was first isolated from the desert plant rhizosphere fungus Aspergillus terreus, has moderate cytotoxicity against four human cancer cell lines (NCIH460, MCF-7, SF-268, and MIA Pa Ca-2) and is a potential anticancer drug (He et al., Journal of Natural Products, 2004, 67(12):1985-1991). Terrequinone A is also the only bis-indolequinone compound with biosynthetic information. In 2004, Bok et al. identified the biosynthetic gene cluster tdiABCDE responsible for the synthesis of terrequinone A by mining secondary metabolome from Aspergillus nidulans using the transcriptional regulator LaeA (Bok et al., Eukaryotic Cell, 2004, 3(2):527-535).

As a natural product, terrequinone A has a complex structure with plurality of functional groups. The chemical synthesis of terrequinone A requires many steps, resulting in low yield and environmental friendliness. With the elucidation of the special pathway for the biosynthesis and modification of terrequinone Ain vivo, biocatalytic synthesis of terrequinone A has become an effective attempt. Balibar et al. successfully synthesized terrequinone A by overexpressing and investigating five genes in gene cluster tdiABCDE and using an enzyme-catalyzed in vitro stepwise reaction (Carl J Balibar et al., Terrequinone A biosynthesis through L-tryptophan oxidation, dimerization and bisprenylation, Nature Chemical Biology, 2007, 3(9):584-592). However, synthesis in vitro requires the exogenous addition of expensive cofactors and cofactor regeneration enzyme systems; moreover, an enzyme-catalyzed product in the free enzymatic catalysis system tends to be the substrate of the next enzyme, and the delivery of substrates and products between enzymes is often limited by spatial constraints, reducing the efficiency of product synthesis. Therefore, a more efficient method is needed to produce terrequinone A. E. coli, as an excellent bioreactor, can be used for the biosynthesis of high-value natural products by modifying synthesis pathways thereof or introducing new metabolic pathways, which can perfectly avoid the above two problems.

### SUMMARY

An objective of the present disclosure is to provide a gene combination for expressing and producing terrequinone A in E. coli and use thereof, thereby solving problems of high production cost and low synthesis efficiency in synthesis in vitro of terrequinone Ain the prior art.

To solve the above technical problems, the present disclosure provides the following technical solutions:

The first aspect of the present disclosure, where a gene combination for expressing and producing terrequinone A in E. coli is provided. The gene combination includes a tdiBS gene, a tdiCS gene, a tdiDS gene, a tdiES gene, an sfpS gene, an ScCKS gene, and an AtIPKS gene, where the tdiAS gene, the tdiBS gene, the tdiCS gene, the tdiDS gene, the tdiES gene, the sfpS gene, the ScCKS gene, and the AtIPKS gene have nucleotide sequences shown in SEQ ID NOS:1 to 8.

The second aspect of the present disclosure, where a recombinant plasmid pC02 for producing terrequinone A is provided. The recombinant plasmid pC02 is constructed by sequentially linking and ligating gene expression cassettes T7 tdiAS, T7 tdiBS, T7 tdiCS, T7 tdiDS, T7 tdiES, and T7 sfpS to an E. coli expression vector, where the gene expression cassettes T7 tdiAS, T7 tdiBS, T7 tdiCS, T7 tdiDS, T7 tdiES, and T7 sfpS are constructed by ligating each of a tdiAS gene, a tdiBS gene, a tdiCS gene, a tdiDS gene, a tdiES gene, and an sfpS gene with nucleotide sequences set forth in SEQ ID NOS:1 to 6 to a T7 promoter and a terminator.

In some embodiments, the E. coli expression vector is pCAMBIA1301.

The third aspect of the present disclosure, where a recombinant plasmid pU03 for producing dimethylallyl diphosphate (DMAPP) desired for terrequinone A synthesis is provided. The recombinant plasmid pU03 is constructed by sequentially linking and ligating gene expression cassettes T7 ScCKS and T7 AtIPKS to an E. coli expression vector, where the gene expression cassettes T7 ScCKS and T7 AtIPKS are constructed by ligating each of an ScCKS gene and an AtIPKS gene with nucleotide sequences set forth in SEQ ID NOS:7 to 8 to a T7 promoter and a terminator.

In some embodiments, the E. coli expression vector is pUC19.

The fourth aspect of the present disclosure, where a preparation method of a recombinant E. coli strain capable of producing terrequinone A, including the following steps: step S1: optimizing a tdiA gene, a tdiB gene, a tdiC gene, a tdiD gene, a tidE gene, an sfp gene, an ScCK gene, and an AtIPK gene according to an E. coli expression pattern to obtain a tdiAS gene, a tdiBS gene, a tdiCS gene, a tdiDS gene, a tdiES gene, an sfpS gene, an ScCKS gene, and an AtIPKS gene with nucleotide sequences set forth in SEQ ID NOS:1 to 8, where the eight genes ligate a T7 promoter and a terminator to construct gene expression cassettes T7 tdiAS, T7 tdiBS, T7 tdiCS, T7 tdiDS, T7 tdiES, T7 sfpS, T7 ScCKS, and T7 AtIPKS; step S2: ligating six gene expression cassettes conducted in step S1, the T7 tdiAS, the T7 tdiBS, the T7 tdiCS, the T7 tdiDS, the T7 tdiES, and the T7 sfpS, to an E. coli expression vector to obtain a recombinant plasmid pC02 containing gene expression cassettes tdiAS, tdiBS, tdiCS, tdiDS, tdiES, and sfpS; step S3: ligating two gene expression cassettes conducted in step S1, the T7 ScCKS and the T7 AtIPKS, to an E. coli expression vector to obtain a recombinant plasmid pU03 containing gene expression cassettes ScCKS and AtIPKS; and step S4: simultaneously transforming the recombinant plasmid pC02 obtained in step S2 and the recombinant plasmid pU03 obtained in step S3 into E. coli to obtain the recombinant E. coli strain capable of producing the terrequinone A.

In step S2, the six gene expression cassettes, namely, the T7 tdiAS, the T7 tdiBS, the T7 tdiCS, the T7 tdiDS, the T7 tdiES, and the T7 sfpS, are sequentially linked and ligated to an EcoRI endonuclease site at a 5′-end of the T7 tdiAS and a HindIII endonuclease site at a 3′-end of the T7 sfpS to obtain EcoRI-T7 tdiAS-T7 tdiBS-T7 tdiCS-T7 tdiDS-T7 tdiES-T7 sfpS-HindIII.

In step S3, the two gene expression cassettes, namely, the T7 ScCKS and the T7 AtIPKS, are sequentially linked and ligated to an EcoRI endonuclease site at a 5′-end of the T7 ScCKS and a HindIII endonuclease site at a 3′-end of the T7 AtIPKS to obtain EcoRI-T7 ScCKS-T7 AtIPKS-HindIII.

The fifth aspect of the present disclosure, a recombinant E. coli strain capable of producing terrequinone A obtained by the foregoing method is provided.

The sixth aspect of the present disclosure, where a method for producing terrequinone A using a recombinant E. coli strain is provided, namely, inoculating the recombinant E. coli strain in M9 liquid medium supplemented with 100 μg/mL ampicillin and 50 μg/mL kanamycin to culture at 37° C. until a bacterial suspension reaches an OD₆₀₀ of 0.6, adding 0.2% arabinose, continuing culturing at 25° C. for 14-18 h to obtain a fermentation broth, adding substrates L-tryptophan and prenol in the fermentation broth, and then culturing at 30° C. for 22-26 h to produce the terrequinone A. Each liter of the M9 liquid medium includes: 15 g of glycerol, 6 g of Na₂HPO₄, 3 g of KH₂PO₄, 1 g of NH₄Cl, 0.5 g of NaCl, 0.12 g of MgSO₄, 0.011 g of CaCl₂), 2.9 mg of ZnSO₄·7H₂O, 0.2 mL of 1% (w/v) vitamin B1, and 5 g of acid-hydrolyzed casein.

An embodiment of the present disclosure, where recombinant E. coli BL-3 is inoculated in optimized M9 liquid medium supplemented with 100 μg/mL ampicillin and 50 μg/mL kanamycin (15 g of glycerol, 6 g of Na₂HPO₄, 3 g of KH₂PO₄, 1 g of NH₄Cl, 0.5 g of NaCl, 0.12 g of MgSO₄, 0.011 g of CaCl₂), 2.9 mg of ZnSO₄·7H₂O, 0.2 mL of 1% (w/v) vitamin B1, and 5 g of acid-hydrolyzed casein per liter), cultured at 37° C. until the bacterial suspension reaches an OD₆₀₀ of 0.6, added with 0.2% arabinose, and kept culturing at 25° C. for 16 h to obtain a fermentation broth; the substrates L-tryptophan and prenol are added to the fermentation broth and cultured at 30° C. for 24 to produce the terrequinone A.

In some embodiments, 0.75 g/L L-tryptophan and 0.95 g/L prenol may be added to the fermentation broth.

In the present disclosure, the tdiA gene, the tdiB gene, the tdiC gene, the tdiD gene, the tidE gene, the sfp gene, the ScCK gene, and the AtIPK gene are optimized as a whole based on the codon preference of E. coli. The optimization principles include: (I) optimizing genes and gene codons to improve the translation efficiency of the genes based on the codon preference of E. coli; (II) eliminating stem-loop structures, transcription termination signals, and reverse repeats within 200 bp of the same or adjacent genes, and balancing the GC/AT content within the genes to improve RNA stability; (III) ensuring that gene encoding proteins comply with the N-end rule to improve the stability of translated proteins; (IV) optimizing the free energy of mRNA secondary structure to improve gene expression efficiency; and (V) on the basis of meeting the above four principles, selecting and eliminating EcoRI and HindIII restriction endonuclease recognition sites within the four genes to facilitate the construction of recombinant plasmids. The tdiAS gene, the tdiBS gene, the tdiCS gene, the tdiDS gene, the tdiES gene, the sfpS gene, the ScCKS gene, and the AtIPKS gene are obtained after optimization.

The present disclosure chemically synthesizes a tdiAS gene encoding nonribosomal peptide synthetase (NRPS), a tdiBS gene encoding indole prenyltransferase, a tdiCS gene encoding oxidoreductase, a tdiDS gene encoding aminotransferase, a tdiES gene encoding chaperone, an sfpS gene encoding phosphopantetheinyl transferase, an ScCKS gene encoding choline kinase, and an AtIPKS gene encoding isopentenyl phosphate kinase. For the first time, the eight gene fragments each are ligated to the T7 promoter and terminator of E. coli to construct the corresponding gene expression cassettes. Six gene expression cassettes, T7 tdiAS, T7 tdiBS, T7 tdiCS, T7 tdiDS, T7 tdiES, and T7 sfpS, are ligated to an E. coli expression vector to obtain a recombinant plasmid pC02. Two gene expression cassettes, T7 ScCKS and T7 AtIPKS, are ligated to an E. coli expression vector to obtain a recombinant plasmid pU03. Both the pC02 and the pU03 are transformed into E. coli to obtain a recombinant engineered strain capable of producing terrequinone A, which has an anti-cancer activity. The content of terrequinone A in a fermentation broth thereof can reach 106.3 mg/L when 0.75 g/L L-tryptophan and 0.95 g/L prenol are added.

Compared with the prior art, the present disclosure has the following beneficial effects:

Using synthetic biology technology, the present disclosure optimizes the tdiA gene, the tdiB gene, the tdiC gene, the tdiD gene, the tidE gene, the sfp gene, the ScCK gene, and the AtIPK gene according to an E. coli expression pattern. After optimization, these genes are separately ligated to the T7 promoter and terminator of E. coli to construct gene expression cassettes. The gene expression cassettes are ligated to the E. coli expression vector to obtain a polygenic E. coli transformation vector to construct a genetically engineered strain, which can express active genes of the tdiAS gene encoding NRPS, the tdiBS gene encoding methylpropanoyl-L-tryptophan synthetase, the tdiCS gene encoding oxidoreductase, the tdiDS gene encoding aminotransferase, the tdiES gene encoding chaperone, the sfpS gene encoding phosphopantetheinyl transferase, the ScCKS gene encoding choline kinase, and the AtIPKS gene encoding isopentenyl phosphate kinase. The genetically engineered strain can also use L-tryptophan and prenol as substrates to produce terrequinone A with anti-cancer activity. which has potential application value in the biopharmaceutical field.

In the terrequinone A synthesis pathway of A. nidulans, synthesis of each terrequione A molecule requires consumption of two DMAPP molecules. The DMAPP produced by original MEP pathway of E. coli cannot meet the demand for terrequione A synthesis. In the present disclosure, a multigene transformation vector pU03 are simultaneously transformed into E. coli to obtain a recombinant strain, which can provide sufficient DMAPP for terrequinone A synthesis using prenol as substrate.

He et al. isolated 6.0 mg of terrequinone A powder from 5.4 L of fermentation broth 28 days after culture of A. terreus (He et al., Journal of Natural Products, 2004, 67(12):1985-1991). Compared with natural strain A. terreus, at 48 h after the culture of the recombinant E. coli strain in the present disclosure, the content of terrequinone A in the fermentation broth can reach 106.3 mg/L, with significant improvement in both transformation rate and product concentration.

### BRIEF DESCRIPTION OF THE DRAWINGS

FIG. 1 schematically illustrates the structure of recombinant plasmids pC02 and pU03 in Example 3 of the present disclosure;

FIG. 2 illustrates a synthesis pathway of terrequinone A by E. coli BL-3 transformation in Example 3 of the present disclosure;

FIG. 3 illustrates a PCR result of exogenous genes of E. coli BL-3 in Example 4 of the present disclosure, where M represents Marker;

FIG. 4 illustrates the mass spectrometry (MS) identification of terrequinone A in Example 5 of the present disclosure (positive ion m/z=491.2);

FIG. 5 illustrates high performance liquid chromatography (HPLC) of a chloroform-extracted bacterial culture medium in Example 5 of the present disclosure; and

FIG. 6 illustrates the content of terrequinone A after addition of substrates in different concentrations in Example 6 of the present disclosure.

### DETAILED DESCRIPTION OF THE EMBODIMENTS

The present disclosure will be further described below with reference to the drawings and specific Examples.

The test methods used in the Examples are conventional molecular biology methods, unless otherwise specified. The materials and reagents used are commercially available, unless otherwise specified.

### Example 1 Optimization of Eight Gene Fragments

Based on coding sequences of tdiA (Genbank: EF550581.1), tdiB (Genbank: EF550582.1), tdiC (Genbank: EF550583.1), tdiD (Genbank: EF550584.1), tidE (Genbank: EF550585.1), sfp (GenBank: X65610.1, Bacillus subtilis), ScCK (GenBank: AAA34499.1, Saccharomyces cerevisiae), and AtIPK (GenBank: AY150412.1, Arabidopsis thaliana) genes, the above eight genes were optimized as a whole according to the following principles:

(I) genes and gene codons were optimized to improve the translation efficiency of the genes based on the codon preference of E. coli; (II) stem-loop structures, transcription termination signals, and reverse repeats within 200 bp of the same or adjacent genes were eliminated and the GC/AT content within the genes are balanced to improve RNA stability; (III) gene encoding proteins was ensured to comply with the N-end rule to improve the stability of translated proteins; (IV) the free energy of mRNA secondary structure was optimized to improve gene expression efficiency; and (V) on the basis of meeting the above four principles, EcoRI and HindIII restriction endonuclease recognition sites within the eight genes were selected and eliminated to facilitate the construction of recombinant plasmids.

The tdiAS gene, the tdiBS gene, the tdiCS gene, the tdiDS gene, the tdiES gene, the sfpS gene, the ScCKS gene, and the AtIPKS gene were obtained after optimization. The tdiAS gene has the nucleotide sequence set forth in SEQ ID NO:1; the tdiBS gene has the nucleotide sequence set forth in SEQ ID NO:2; the tdiCS gene has the nucleotide sequence set forth in SEQ ID NO:3; the tdiDS gene has the nucleotide sequence set forth in SEQ ID NO:4; the tdiES gene has the nucleotide sequence set forth in SEQ ID NO:5; the sfpS gene has the nucleotide sequence set forth in SEQ ID NO:6; the ScCKS gene has the nucleotide sequence set forth in SEQ ID NO:7; and the AtIPKS gene has the nucleotide sequence set forth in SEQ ID NO:8.

### Example 2 Construction of Gene Expression Cassettes

T7 promoter sequence of E. coli was ligated at the front end of each optimized gene sequence and T7 terminator sequence of E. coli was ligated at the end of each optimized gene to construct gene expression cassettes T7 tdiAS, T7 tdiBS, T7 tdiCS, T7 tdiDS, T7 tdiES, T7 sfpS, T7 ScCKS, and T7 AtIPKS, which were chemically synthesized by Nanjing GenScript Biotech Corporation.

The T7 promoter sequence
(SEQ ID NO: 9) was:
5′-TAATACGACTCACTATAGG-3′;
The T7 terminator sequence
(SEQ ID NO: 10) was:
5′-TAGCATAACCCCTTGGGGCCTCTA
AACGGGTCTTGAGGGGTTTTTTG-3′.

### Example 3 Construction and Transformation of Recombinant Plasmids

The “polyacrylamide gel electrophoresis (PAGE)-mediated gene splicing by overlap extension PCR” technology (Peng R H et al., A direct and efficient PAGE-mediated overlap extension PCR method for gene multiple-site mutagenesis, Appl Microbiol Biotechnol. 2006,73(1):234-40) was used to sequentially link and ligate the six constructed gene expression cassettes, T7 tdiAS, T7 tdiBS, T7 tdiCS, T7 tdiDS, T7 tdiES, and T7 sfpS, to the EcoRI endonuclease recognition site at the 5′-end of the T7 tdiAS and the HindIII endonuclease recognition site at the 3′-end of the T7 sfpS to obtain EcoRI-T7 tdiAS-T7 tdiBS-T7 tdiCS-T7 tdiDS-T7 tdiES-T7 sfpS-HindIII, and to sequentially link and ligate the two gene expression cassettes, T7 ScCKS and T7 AtIPKS, to the EcoRI endonuclease recognition site at the 5′-end of the T7 ScCKS and the HindIII endonuclease recognition site at the 3′-end of the T7 AtIPKS to obtain EcoRI-T7 ScCKS-T7 AtIPKS-HindIII.

In order to obtain EcoRI-T7 tdiAS-T7 tdiBS-T7 tdiCS-T7 tdiDS-T7 tdiES-T7 sfpS-HindIII, the designed primer sequences (SEQ ID NOS:11 to 22) were as follows:

T7 tdiAS:
P1:
(SEQ ID NO: 11)
5′-GAATCCTAATACGACTCACTATA
GGATGGCACCATCTAAG-3′;
P2:
(SEQ ID NO: 12)
5′-GTATTCAGTAGCCATCCTATAGTGAGTC
GTATTACAAAAAACCCCTC-3′;
T7 tdiBS:
P3:
(SEQ ID NO: 13)
5′-TTGAGGGGTTTTTTGTAATACGACTCAC
TATAGGATGGCTACTGAATAC-3′;
P4:
(SEQ ID NO: 14)
5′-AAGAGCTGCGTGCATCCTATAGTGAGTC
GTATTACAAAAAACCCCTC-3′;
T7 tdiCS:
P5:
(SEQ ID NO: 15)
5′-TTGAGGGGTTTTTTGTAATACGACTCAC
TATAGGATGCACGCAGCTCTT-3′;
P6:
(SEQ ID NO: 16)
5′-ACCAATAGAACCCATCCTATAGTGAGTC
GTATTACAAAAAACCCCTC-3′;
T7 tdiDS:
P7:
(SEQ ID NO: 17)
5′-TTGAGGGGTTTTTTGTAATACGACTCAC
TATAGGATGGGTTCTATTGGT-3′;
P8:
(SEQ ID NO: 18)
5′-ATGGTCTGTTAACCATCCTATAGTGAGT
CGTATTACAAAAAACCCCTC-3′;
T7 tdiES:
P9:
(SEQ ID NO: 19)
5′-TTGAGGGGTTTTTTGTAATACGACTCAC
TATAGGATGGTTAACAGACCAT-3′;
P10:
(SEQ ID NO:20)
5′-ACCGTAGATTTTCATCCTATAGTGAGTC
GTATTACAAAAAACCCCTC-3′;
T7 spfS:
P11:
(SEQ ID NO: 21)
5′-TTGAGGGGTTTTTTGTAATACGACTCAC
TATAGGATGAAAATCTACGGT-3′;
and
P12:
(SEQ ID NO: 22)
5′-AAGCTTCAAAAAACCCCTCAAGACCCGT
TTAGAGG-3′.

First-step PCR: PCR amplification was conducted using gene fragments of T7 tdiAS, T7 tdiBS, T7 tdiCS, T7 tdiDS, T7 tdiES, and T7 sfpS as templates separately, and adding the corresponding primers to obtain six gene fragments. The PCR amplification program was as follows: 94° C. for 30 s, 68° C. for 60 s, for a total of 10 cycles.

The six gene fragments obtained in the first-step PCR were subjected to gel electrophoresis and gel extraction.

Second-step PCR: PCR amplification was conducted using a mixture of the six gene fragments obtained by gel extraction as a template and P1 and P12 as primers. The PCR amplification program was as follows: initial denaturation at 94° C. for 1 min; 25 cycles of denaturation at 94° C. for 30 s, annealing at 58° C. for 30 s, and extension at 72° C. for 30 s; and finally extension at 72° C. for 10 min. In order to obtain EcoRI-T7 tdiAS-T7 tdiBS-T7 tdiCS-T7 tdiDS-T7 tdiES-T7 sfpS-HindIII, the designed primer sequences (SEQ ID NOS:23 to 26) were as follows:

T7 ScCKS:
P13:
(SEQ ID NO: 23)
5′-GAATCCTAATACGACTCACTATAGGAT
GGTTCAAGAGT-3′;
P14:
(SEQ ID NO: 24)
5′-CTTCTCCAGCTCGTTCCCTATAGTGAG
TCGTATTACAAAAAACCCCTC-3′;
T7 AtIPKS:
P15:
(SEQ ID NO: 25)
5′-TTGAGGGGTTTTTTGTAATACGACTCA
CTATAGGGAACGAGCTGGAGAAG-3′;
and
P16:
(SEQ ID NO: 26)
5′-AAGCTTCAAAAAACCCCTCAAGACCCG
TTTAGAGG-3′.

First-step PCR: PCR amplification was conducted using gene fragments of T7 ScCKS and T7 AtIPKS as templates separately, and adding the corresponding primers to obtain two gene fragments. The PCR amplification program was as follows: 94° C. for 30 s, 68° C. for 60 s, for a total of 10 cycles.

The two gene fragments obtained in the first-step PCR were subjected to gel electrophoresis and gel extraction.

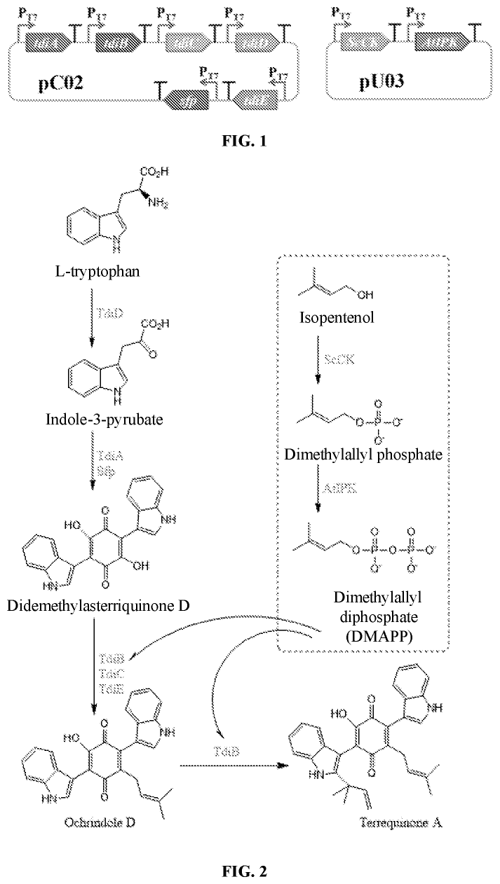

Second-step PCR: PCR amplification was conducted using a mixture of the two gene fragments obtained by gel extraction as a template and P13 and P16 as primers. The PCR amplification program was as follows: initial denaturation at 94° C. for 1 min; 25 cycles of denaturation at 94° C. for 30 s, annealing at 58° C. for 30 s, and extension at 72° C. for 30 s; and finally extension at 72° C. for 10 min. The above tandem six-gene fragment EcoRI-T7 tdiAS-T7 tdiBS-T7 tdiCS-T7 tdiDS-T7 tdiES-T7 sfpS-HindIII was double digested with EcoRI and HindIII and ligated to a vector, pCAMBIA1301, obtained by the same digestion to obtain a recombinant plasmid pC02 containing six genes; the two-gene fragment EcoRI-T7 ScCKS-T7 AtIPKS-HindIII was double digested with EcoRI and HindIII and ligated to a vector, pUC19, obtained by the same digestion to obtain a recombinant plasmid pU03 containing two genes (as shown in FIG. 1).

Both pC02 and pU03 were simultaneously transformed into E. coli BL21-AI by the heat shock method, spread on an LB solid plate supplemented with 100 μg/mL ampicillin and 50 g/mL kanamycin, and cultured at 37° C. overnight to pick the positive clone, namely recombinant E. coli BL-3. The synthesis pathway of terrequinone A by its transformation is shown in FIG. 2.

### Example 4 Identification of Recombinant E. coli Strain

Successful transformation of exogenous genes into E. coli was identified by PCR. A single colony was picked and inoculated in LB liquid supplemented with 100 μg/mL ampicillin and 50 μg/mL kanamycin, cultured at 37° C. until the bacterial suspension reached an OD₆₀₀ of 0.6, and centrifuged at 10,000 rpm for 1 min at 4° C. to collect cells. Plasmid DNA was extracted from the cells by the Trizol method; using extracted plasmid as a PCR template, exogenous tdiAS, tdiBS, tdiCS, tdiDS, tdiES, sfpS, ScCKS, and AtIPKS genes were detected by PCR using the following primers and amplification conditions.

Sequences of primers used for PCR (SEQ ID NOS:27 to 42) were as follows, where the primer were designed based on specific fragments of each gene:

tdiA-F:
(SEQ ID NO: 27)
5′-TCCGTCAAGTGCATGGATGTC-3′;
tdiA-R:
(SEQ ID NO: 28)
5′-CAGACCACGCTCACGCAGGAC-3′;
tdiB-F:
(SEQ ID NO: 29)
5′-GCACTGAAGAAGCTGGGTAAC-3′;
tdiB-R:
(SEQ ID NO: 30)
5′-ACGGAAACCGAAGTCACCAGC-3′;
tdiC-F:
(SEQ ID NO: 31)
5′-ATCTCTCGTAAGCCAATCTGC-3′;
tdiC-R:
(SEQ ID NO: 32)
5′-GACGATGACGACACGGGAACC-3′;
tdiD-F:
(SEQ ID NO: 33)
5′-ATGTTCGTCTGGCTGGAACTC-3′;
tdiD-R:
(SEQ ID NO: 34)
5′-GCAACGATCGACCAGACCAGC-3′;
tdiE-F:
(SEQ ID NO: 35)
5′-AAGACCTTGGGTTTGTGGAAC-3′;
tdiE-R:
(SEQ ID NO: 36)
5′-GACGTCGGAACCAGGTGCAGC-3′;
sfp-F:
(SEQ ID NO: 37)
5′-TCTCACTCTGGACGTTGGGTG-3′;
sfp-R:
(SEQ ID NO: 38)
5′-TGCAGATGCGATGAGACGTTG-3′;
ScCK-F:
(SEQ ID NO: 39)
5′-GGTCCTAACGGCAAGAAGTAC-3′;
ScCK-R:
(SEQ ID NO: 40)
5′-GGAGGAGAAGTTCTTGCACTC-3′;
AtIPK-F:
(SEQ ID NO: 41)
5′-CCACTGTTGGAGCACACTGAC-3′;
and
AtIPK-R:
(SEQ ID NO: 42)
5′-GGAGAAACGGATGATAGTACC-3′.

The amplification program used was as follows: initial denaturation at 94° C. for 3 min; 30 cycles of denaturation at 94° C. for 30 s, annealing at 54° C. for 30 s, and extension at 72° C. for 30 s; and finally extension at 72° C. for 10 min. The results are shown in FIG. 2.

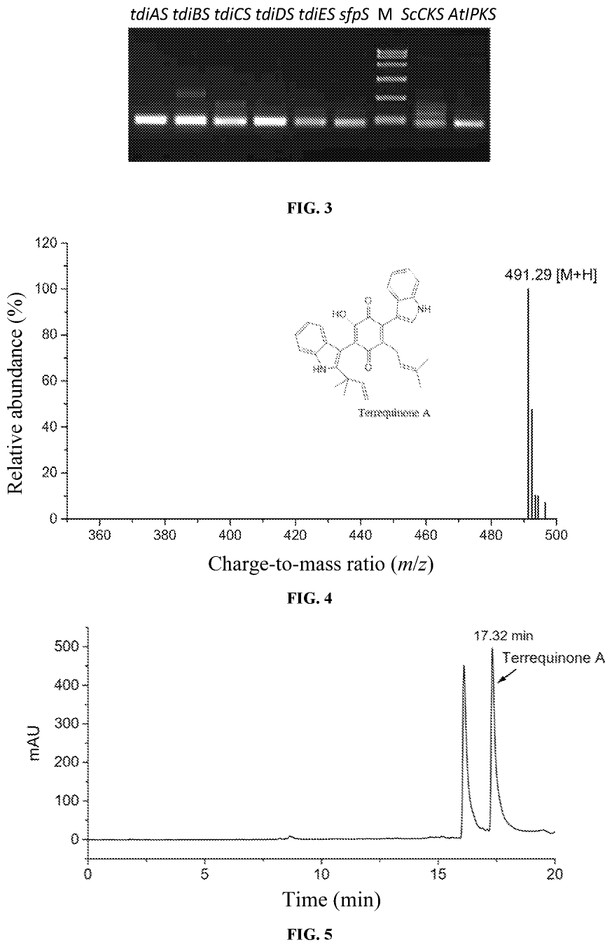

The results in FIG. 3 showed that the recombinant E. coli BL-3 provided by the present disclosure could amplify the above eight genes, tdiAS, tdiBS, tdiCS, tdiDS, tdiES, sfpS, ScCKS, and AtIPKS, indicating that both recombinant plasmids, pC02 and pU03, were transformed into E. coli and the obtained recombinant E. coli BL-3 included the eight exogenous genes.

### Example 5 Production of Terrequinone a by Recombinant E. coli BL-3

A single colony transformed with polygenic recombinant plasmids pC02 and pU03 in Example 3 of the present disclosure was picked and inoculated in optimized M9 liquid medium supplemented with 100 μg/mL ampicillin and 50 μg/mL kanamycin (15 g of glycerol, 6 g of Na₂HPO₄, 3 g of KH₂PO₄, 1 g of NH₄Cl, 0.5 g of NaCl, 0.12 g of MgSO₄, 0.011 g of CaCl₂), 2.9 mg of ZnSO₄·7H₂O, 0.2 mL of 1% (w/v) vitamin B1, and 5 g of acid-hydrolyzed casein per liter). The single colony was cultured at 37° C. until the bacterial suspension reached an OD₆₀₀ of 0.6. 0.2% L-arabinose (final concentration) was added, and the culture was continued at 25° C. for 16 h to obtain a fermentation broth. 0.5 g/L L-tryptophan and equal amount of substance of prenol were added to the fermentation broth and cultured at 30° C. for 24 h.

200 μL of fermentation broth was added with twice the volume of methanol to broken cells by ultrasound (ultrasound is conducted at a power of 400 W for 4 s, at an interval of 8 s, repeated for 99 rounds). After centrifugation at 10,000 rpm for 1 min at 4° C., the supernatant was collected, extracted with an equal volume of chloroform, evaporated under reduced pressure, and reconstituted in methanol for terrequinone A detection.

Through liquid chromatography mass spectrometry (LC-MS), the mass spectrum of the sample (as shown in FIG. 4) was compared with the reported mass spectrum of terrequinone A in literature (Balibar et al., Nature Chemical Biology, 2007, 3(9):584-592), and the sample was identified as terrequinone A. The specific LC-MS conditions were as follows: TSQ Quantum-Accela LC-MS System; Shimadzu Shim-pack GIST C18 column (150×2.1 mm, 3 m); mobile phase A of 0.1% formic acid in water, and mobile phase B of 0.1% formic acid in acetonitrile were used. The LC-MS is conducted by gradient elution: 0-3 min: 20% B; 3-13 min: 20% B to 90% B; with a flow rate of 0.2 μL/min, a column temperature of 35° C., a sampling volume of 1 μL, an ion source in ESI+ mode, an electrospray voltage of 3,500 V, an sheath gas flow rate of 13 mL/min, an ion transfer tube temperature of 275° C., a scan mode of full scan mode, a scan resolution of 0.4 Da, an acquired mass-to-charge ratio range of m/z=300-500 Da, a scan time of 0.5 s.

The content of terrequinone A was determined by HPLC. Specific HPLC detection conditions were as follows: Agilent 1100 High Performance Liquid Chromatography System, C18 column (120 A, 4.6×150 mm, 5 μm), mobile phase of 0.1% trifluoroacetic acid-acetonitrile-water were used. And the HPLC is conducted by gradient elution from 5% to 100% in 20 min with a flow rate of 1 mL/min, a column temperature of 35° C., a detection wavelength of 280 nm and an sampling volume of 20 μL. The determined content of terrequinone A was 41.9 mg/L (FIG. 5).

### Example 6 Effect of Substrate Addition on Production of Terrequinone a by Recombinant E. coli BL-3

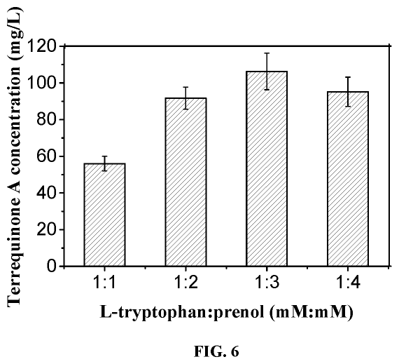

A single colony of recombinant E. coli BL-3 obtained in Example 3 was picked, inoculated in 50 mL of optimized M9 liquid medium supplemented with 100 μg/mL ampicillin and 50 μg/mL kanamycin. The single colony was cultured at 37° C. until the bacterial suspension reached an OD₆₀₀ of 0.6. 0.2% arabinose was added, and the culture was continued at 25° C. for 16 h to obtain a fermentation broth. 0.75 g/L L-tryptophan and prenol of different concentrations (0.32, 0.63, 0.95, and 1.27 g/L) were added to the fermentation broth to make the molar ratio of L-tryptophan and prenol reach 1:1, 1:2, 1:3, and 1:4, and cultured at 30° C. for 24 h. The content of terrequinone A in BL-3 fermentation broth was determined under different substrate addition. As shown in FIG. 6, when the molar ratio of L-tryptophan and prenol was 1:3 (0.75 g/L L-tryptophan and 0.95 g/L prenol), the terrequinone A in the fermentation broth of the recombinant E. coli strain herein could reach 106.3 mg/L; however, the terrequinone A powder could be separated from 5.4 L of fermentation broth after culturing natural strain A. terreus for 28 days was 6.0 mg. According to the results, the recombinant E. coli BL-3 provided by the present disclosure shows significant improvement in both transformation rate and terrequinone A product concentration.

The above are only the preferred examples of the present disclosure and are not intended to limit the scope of the present disclosure. Various changes can also be made to the above examples of the present disclosure. All simple and equivalent changes and modifications made in accordance with the claims and specification of the present disclosure fall within the claimed scope of the present disclosure. All that is not described in detail in the present disclosure is conventional technical content.

What is claimed is:

1. A recombinant plasmid composition for producing terrequinone A, comprising recombinant plasmid pC02 and recombinant plasmid pU03, wherein the recombinant plasmid pC02 is constructed by sequentially linking and ligating gene expression cassettes T7 tdiAS, T7 tdiBS, T7 tdiCS, T7 tdiDS, T7 tdiES, and T7 sfpS to an Escherichia coli expression vector, wherein the gene expression cassettes T7 tdiAS, T7 tdiBS, T7 tdiCS, T7 tdiDS, T7 tdiES, and T7 sfpS are constructed by ligating each of a tdiAS gene, a tdiBS gene, a tdiCS gene, a tdiDS gene, a tdiES gene, and an sfpS gene with nucleotide sequences set forth in SEQ ID NOS:1 to 6 to a T7 promoter and a terminator; wherein the recombinant plasmid pU03 for producing dimethylallyl diphosphate (DMAPP) desired for terrequinone A synthesis, wherein the recombinant plasmid pU03 is constructed by sequentially linking and ligating gene expression cassettes T7 ScCKS and T7 AtIPKS to an Escherichia coli expression vector, wherein the gene expression cassettes T7 ScCKS and T7 AtIPKS are constructed by ligating each of an ScCKS gene and an AtIPKS gene with nucleotide sequences set forth in SEQ ID NOS:7 to 8 to a T7 promoter and a terminator.

2. The recombinant plasmid pC02 according to claim 1, wherein the Escherichia coli expression vector is pCAMBIA1301.

3. The recombinant plasmid pU03 according to claim 1, wherein the Escherichia coli expression vector is pUC19.

4. A preparation method of a recombinant Escherichia coli strain capable of producing terrequinone A, comprising the following steps:
step S1: optimizing a tdiA gene, a tdiB gene, a tdiC gene, a tdiD gene, a tidE gene, an sfp gene, an ScCK gene, and an AtIPK gene according to an Escherichia coli expression pattern to obtain eight genes, namely, a tdiAS gene, a tdiBS gene, a tdiCS gene, a tdiDS gene, a tdiES gene, an sfpS gene, an ScCKS gene, and an AtIPKS gene with nucleotide sequences set forth in SEQ ID NOS:1 to 8, ligating each of the eight genes to a T7 promoter and a terminator to construct gene expression cassettes T7 tdiAS, T7 tdiBS, T7 tdiCS, T7 tdiDS, T7 tdiES, T7 sfpS, T7 ScCKS, and T7 AtIPKS;
step S2: sequentially linking and ligating six gene expression cassettes obtained in step S1, namely, the T7 tdiAS, the T7 tdiBS, the T7 tdiCS, the T7 tdiDS, the T7 tdiES, and the T7 sfpS, to an Escherichia coli expression vector to obtain a recombinant plasmid pC02 comprising six gene expression cassettes tdiAS, tdiBS, tdiCS, tdiDS, tdiES, and sfpS;
step S3: sequentially linking and ligating two gene expression cassettes obtained in step S1, namely, the T7 ScCKS and the T7 AtIPKS, to an Escherichia coli expression vector to obtain a recombinant plasmid pU03 comprising two gene expression cassettes ScCKS and AtIPKS; and
step S4: simultaneously transforming the recombinant plasmid pC02 obtained in step S2 and the recombinant plasmid pU03 obtained in step S3 into Escherichia coli to obtain the recombinant Escherichia coli strain capable of producing the terrequinone A.

5. The preparation method of a recombinant Escherichia coli strain capable of producing terrequinone A according to claim 4, wherein in the step S2, the six gene expression cassettes, namely, the T7 tdiAS, the T7 tdiBS, the T7 tdiCS, the T7 tdiDS, the T7 tdiES, and the T7 sfpS, are sequentially linked and ligated to an EcoRI endonuclease site at a 5′-end of the T7 tdiAS and a HindIII endonuclease site at a 3′-end of the T7 sfpS to obtain EcoRI-T7 tdiAS-T7 tdiBS-T7 tdiCS-T7 tdiDS-T7 tdiES-T7 sfpS-HindIII.

6. The preparation method of a recombinant Escherichia coli strain capable of producing terrequinone A according to claim 4, wherein in the step S3, the two gene expression cassettes, namely, the T7 ScCKS and the T7 AtIPKS, are sequentially linked and ligated to an EcoRI endonuclease site at a 5′-end of the T7 ScCKS and a HindIII endonuclease site at a 3′-end of the T7 AtIPKS to obtain EcoRI-T7 ScCKS-T7 AtIPKS-HindIII.

7. A recombinant Escherichia coli strain capable of producing terrequinone A obtained by the preparation method according to claim 4.

8. The recombinant Escherichia coli strain capable of producing terrequinone A of claim 7, wherein in the step S2, the six gene expression cassettes, namely, the T7 tdiAS, the T7 tdiBS, the T7 tdiCS, the T7 tdiDS, the T7 tdiES, and the T7 sfpS, are sequentially linked and ligated to an EcoRI endonuclease site at a 5′-end of the T7 tdiAS and a HindIII endonuclease site at a 3′-end of the T7 sfpS to obtain EcoRI-T7 tdiAS-T7 tdiBS-T7 tdiCS-T7 tdiDS-T7 tdiES-T7 sfpS-HindIII.

9. The recombinant Escherichia coli strain capable of producing terrequinone A of claim 7, wherein in the step S3, the two gene expression cassettes, namely, the T7 ScCKS and the T7 AtIPKS, are sequentially linked and ligated to an EcoRI endonuclease site at a 5′-end of the T7 ScCKS and a HindIII endonuclease site at a 3′-end of the T7 AtIPKS to obtain EcoRI-T7 ScCKS-T7 AtIPKS-HindIII.


SEQUENCE LISTING (42 sequences)

SEQ ID NO: 1 - DNA, 2913 bp, synthetic construct
  source 1..2913: mol_type = other DNA; note = Nucleotide sequence of tdiAS gene
atggcaccatctaagactgaaattgcaccacttagagcagcaaaatatccatttggtaacattgttgatgctcttcgtcatgctgctgcacataccgatgagggtatcatcgtctaccatccaaactccatctccacctcctccccaccacagaccgtctcctacaaagacctgctgcatcaggctgaggcaaacgcaactcgtctgttgcagcagaagctgtgttctccaaagtccatcgtcctggttcacttcgagtctgcactggactccatcgtctggtactggtctgtccttctggctggtggtattcctgcactgactggtcctggtatgttctctcagaaccctgctgatcgtgaacgtcatctgcgtcatctgtctgagaccctgaactctcctgtctgtctgactcgtcctgcactgttggcaccatttgaagagcagactgctgatgaccgtatcaaagctcgtactgtcgatgagatcctggctgcacctgaaatcgctgatgttgctgacgcaccactgcctgcactgactccatcttctactgacatgcttgcactgatgctgacttctggttcttctggtaatgccaaggctgttccactgactcatcaacagctgcttgctgcctttcgtggtaagtctactgctgcttctctgcgtttcccacgttctcctttcctgtcttgggttcacatggatcatgttgctaacctggttcattgccacatcttcgctatcgtttctggtatctcccagatccaagttcctgcacctgacctgctgatcaaccctgcacagcttctgaacctgatctctcgtcaccgtgtctctcgtaccttcatgccaaacttcctgtgtgccaagttgcgtcgtcagctggagtctggttctccagagtacatccttgaccctggtctgaacctggaaaccctgtacatcgatactggtggtgaggctaacgtcactgaggtctgcattgcactgcaatctctgctgtctcgttacggtgctcctgacaacgtcttcaagccatccttcggtatgactgagactgttgctggttgcatcttcaactctcattgtccatcctatgaccatgcacaacgtcatgagttcgcatgtcttggtaagcctatgcctggtgttcgtatgcgtgtcactcgtctggatactccatctgaagaggctgctcctggtgaacgtggttctctggaagtcactggtgaagttgtcttcaagggttactacaacaaccctgctgcaactgctgaggcattcacctctgatggttggttccgtactggtgatctggcattcattgactccaatggtaatctgcatcttgatggtcgtactaaggagatgatcaacatcaatggtgtcaaatacctgccatacgaactggatgctgctcttgagcaggcacagattcctggtgcaactccatcttacttctgcaccttctcctctcgtgatgcaactatggacactgaggttgtcgttgtcctgtaccttccatcctacgtcgagtctgatgatgaagcacgtttctctactcaatcctctatcatccgtgttgttgcaatgcatactcgttctcgtccacgtgtcgttccactgcgtccacaggacatgccaaagtccactctgggtaagctgtctcgtgcaaagctgaagactgcactggaagaaggtcagttcgcaactcaacaacaaatcaacgacgaagccatccgtcgttatcagcagaagactcgtgcatctcctgaaactcctgacgaagctgtcattctggatattatcaaggaacagctggagatccggtctgatgatgacagctttggcgtcaacgattcaatcctgtccatcggtgcaacctctatggatctggttgcaatcattcaccgtatcaacaagtgtcttcagccatctcagccactgcgtctgactgacatcctgaaagactccactgctcgtggtctggctgttgcacttgcaactggtgctgcaccacgttctcaggatcagtcttctactcatgtctacgatcctgttgtcactcttcaaccacatggtactaagtctccactgtggctggttcatcctggtgttggtgaggtccttgtcttcgtcaaccttgcacaccacatcaccgaccgtcctgtctacgcattccgtgcaaagggtttcaatgctgctgctggtctgcctgagactccattcacctctcttgaggagttgttcactacctaccgtgatgcaatcaaggaacgtcaaccacatggtccatacgcaatcgctggttactccttcggtggtatggttgcattcgaagtctccaagctgcttgaacaggatggtgatgaggttcgttactgtggttcttggaaccttccaccacacatcaagttccgtatgcgtgaactcgtctgggaggaatgcgttatccatctattctacttcgttggtctgatgactgaactggctgcatacactcacaaaccaactcttcaggagttcaaccgtgcaaaccgtcgtctggatgctatccgttacctgcgtcaacactgtgatgctgcacgttgggatgaacttggtctgtctgaggagtactatctgctgtgggttggtctggcatctaacatgcaatcccttgctgttgactacgaaccatctggttccgtcaagtgcatggatgtctttgttgctgatccattgtctcatgttgcaaaggaccgtatcgactgggttgagggtcgtttgtctgcatggaaggagttcgttcgtgaggatgtccgtttccatgatgtccaaggtgcacactacaccatgctgaaccgtgagtacgtcgaggcattcgctggtactctgaagaacgtcctgcgtgagcgtggtctgtaa

SEQ ID NO: 2 - DNA, 1260 bp, synthetic construct
  source 1..1260: mol_type = other DNA; note = Nucleotide sequence of tdiBS gene
atggctactgaatactggtctcgtcatcttcgttctgttcttgcaccactgttcgctgctgctggtacttactctcctgaagatcaggagtctcatctggccttcattgacgagcacattgcaccaaacctgggtccactgccttgggaaccacatggtccatactctactccatcttctcttgttggttctccattcgatccatctatcaacatcgtctcttctggtaaggccaaggttcgtttcgacttcgatgtcatctctccacctgatcgtactggtcctgacccattcgctgagggatctgctcgtgagatccttcatcgtcttgctgaccttgttggtgctgacactcagtggatgggttatctgatggatgctctgtacctgactcctgctgaggctgaggttgccaaaaccaagctgccaccaggtgttgccattccaccatcttctgttggtttcgatttcgatggtcctgaacgtactctgaagttctacatcccatctgttcgtaaggcattggcaactggtcaggatgtctctgaactcatgctgaagactctgcgtggtttgcagccacttggttctgaactcgttcctgctatggatctgattgcctcttacctgtccactcgtaccaacgacgctatgcttccacttgttggtatcgactgtcttgatccacgtactcacaagaacgctcgtgtcaaatgctacctgcacacctcttccaactctttcgctgttgttcgtgatgtcctgactcttggtggtcgtctgtctgatgatacctctctgaagcgtgttgagactctgaaatccgtttggccactgctgatcaacgaactcgaaggtccacagtctgatgctgctactatggatgagtcctggtccaagcctgaacgtctgaaccgtactggttactctggtatccagtacaccatcgagatcactcctggtcaggcaatccctgataccaagatttacgttccattgttccagtacaccgactcttctgaggttgctgagcgtaacttcgagtctgcactgaagaagctgggtaacgaatggggtctgtctggtaagtaccgttctgttatgcaggaaatcttcaaggatgtcgagaactacggtcagacctacgcatccttctcctacaccgagggtaagggtgtctacaccacctcctacgtcgctatgccaatcaaggatgagggtggtggttccctggctggtgacttcggtttccgtaactaa

SEQ ID NO: 3 - DNA, 1068 bp, synthetic construct
  source 1..1068: mol_type = other DNA; note = Nucleotide sequence of tdiCS gene
atgcacgcagctcttgttccaacttggtcttctccatgtccaatctatactgaaattccagatcctggtccaccaccaccagaacagctgcaactgaaagtcttggctgttggtatcccacgtgttgttcgtcttcgtgctcgtggtatccatcctactgccaagtctgcctctcttccatacgatccatccattgatggtgttggtatcgacgaacaaaccggtatcatgtactacatccttccactctctgcctcttgtctggctgagaaagtcaacgttgaccgtgacaaccttgttccacttcagcctggtgcaccaaaaccacaaccacgtaacggtcctgagaacggttacggtatcgcacttggtgacgctgctgatcatcgtgctgagactctggacccaatcgcaattgctggtctggtcaatcctgtctcctcttcttggatggcattgcgtactcgtgttgatggtgagatcactggtaagactgttctggttcttggtgctacttccaagtctggtcgtgctgctgttctcgttgcacgtttcctgggtgccaacaaagtcatcggtgttgcacgtcgtgaggaaggtctgcgttctgtcgaaggtctggatggttgggtcacttctggtgacatgcttcctggtgagactggtgttcgtttcgcacttcctgactgggttggtcctgtccacatcgtcctggactacgttggtggttctgttgctgctggtgttcttggttctgctgagatcgaggagggtcgtgagttgcaatacgtccaggttggtaatctggcacttgagcttggtactggtgagaagcacatgttcgagaccttgcctggtcatctgatctctcgtaagccaatctgcattcgtggttctggtatgggttctttctctcgtcgtgatctggttcgtgagatgcctggtttggttgcattccttgctcgtatgaaggcaccattcggtatcgcatctgctccaatgtgtgaggttgcatccgtctggcaggacgaggacaccaagggttcccgtgtcgtcatcgtcccttaa

SEQ ID NO: 4 - DNA, 1311 bp, synthetic construct
  source 1..1311: mol_type = other DNA; note = Nucleotide sequence of tdiDS gene
atgggttctattggtgctaacaatgctgttgctgatccaactccacttttctcttccagagttcagaaatgggaacctggtgcaatccgttctcttcttccactggaagcccttcctggtatgatctccctggttgctggtaagccatctcctgagaccttcccaatcgctgagatcgctatctctctgaaggacactcctgctggtactggtcgtatcgttgttgatggtgatgaactgaaccaggctctgcaatacggtcttcctcgtggtaacgcacagctcatccagtggttcgagtctcttcagcgttctgttcatggtctggacgagaatggtggttggtcttgttgcatcggtaacggttctcaggagctgatccatcgtgtcatccaggtcttcactgaccctggtgatccagttctgctggaaactcctgcataccctggtgttgctggtttccttcgtgctgatggtcaggagctgatccctgtctactctgacgctcagggtctgaatcctgcctctctggaacaggcactgtctgaatggcctggtgactctccacgtcctaaggtcctgtacaccactccaactggttccaacccaaccggtcaatcttgcactgagtctcgtaaggctgagatcctgcgtcttgccaagcgtttcaacttcatcatcctggaagacgatgcctactactacctgaactacggtgacgacaaacagcgtgcacgttcctaccttgctctggaacgtgacgtcaacggtgagtctggtcgtgtcgttcgtttcgattctctgtccaagatcgtctctcctggtatgcgtcttggtatcctgactgcacaagctgctgttgttgacaaggttgttcgtatcactgagaacatcaaccttcaaccatcctccaccacccaactgcttgctctgtcccttcttcgtcactggggtcaagctggtttcctgaagcactgtgctgaagctgctgaggtctaccgtcgtcgtcgtgacgtcttcgtctctgctgctgagcgtcaccttcagggtcgtgctacttgggtcgttccaactgctggtatgttcgtctggctggaactcaagttgccacctgagatggactccttcgagcttctgaagtcccagggtatgaagaacggtgttctcgctatccctggtgttgccttcatgcctggtaacgaacagacctgctacatccgtgtctccttctctctcgttcctgagcgtgatatggatgaggcatgtcgtcgtatcgctggtctggtcgatcgttgcgcctgccactcctaa

SEQ ID NO: 5 - DNA, 953 bp, synthetic construct
  source 1..953: mol_type = other DNA; note = Nucleotide sequence of tdiES gene
atggttaacagaccatacgcaaatatgaatgatcttgctaatgctgttgctgaatctgttcagaaatatctggacaactctggtcaagccagaaacggtctgttccagaaaaccaacaacggtgaacgtgacatcgaaaccgagcgtctggcactccagcaccagctcttccacctgactctggacggtaaactccagttgtctccactgccttctccagttcaatccgttctggacatcgccactggtgactccacctgggtccatgccttcgctgaacagaacccatctgcctacatcgttgccaacgacccatctccaacctccaagatcccacttggtctgtctgtcatcccagacgcttccgatgccaacgaaccttggacctacaccagacagttcgacttcgtccactgtagacagcaccatcgtcgtcttgacgaaccacgtctgttcaagcaggcattctctttctgactcctggtggttggctggagatgcaggaactctccaaccctgtcacctctgatgacggtactctgtccgagaacaacccactgtctcagtggggtcgtcttctgatcgaggcatccaagaagatgaaccgtcctgtcgataaccctgccaaatacgagacctggatgcgtgaggctggtttcgtcaactgccacaccgttgcctacaactggcctaccaacccttggcctgctgacgagaagggtaagaccttgggtttgtggaacctgtacaacgtcttgcagcgtttcgaagagttctctgtcgccttgttggtcaaagtcctcggttgggagatggacgatgctaagaccttcttgggtaacgtcaaggaggagctgatgaacgagggtgttcatggttactggcctgtctacgttgtctacggtcagaagccagctgcacctggttccgacgtcatcaccgactccgagtaa

SEQ ID NO: 6 - DNA, 498 bp, synthetic construct
  source 1..498: mol_type = other DNA; note = Nucleotide sequence of sfpS gene
atgaaaatctacggtatctacatggatcgtccactctcacaggaggagaacgaacgtttcatgtcattcatctcacctgagaagcgtgagaagtgtcgtcgtttctatcacaaggaagatgcacatcgtactctgctgggtgacgtcctggttcgttctgtgatctcacgtcagtaccagttggacaagtctgacatccgtttctcaactcaggagtatggtaagccatgcattcctgacctgcctgatgcacacttcaacatctctcactctggacgttgggtgatctgtgcattcgactcacagccaatcggaatcgacatcgagaagaccaagccaatctcacttgagatgcctaagcgtttcttctccaagactgagtactctgacctgcttgccaaggacaaggacgagcagaccgactacttctaccatctctggtccatgaaggaatccttcatcaagcaaggacgtcaacgtctcatcgcatctgcataa

SEQ ID NO: 7 - DNA, 1749 bp, synthetic construct
  source 1..1749: mol_type = other DNA; note = Nucleotide sequence of ScCKS gene
atggttcaagagtcacgtcctggttctgttcgttcatactctgttggctaccaagcacgttcacgttcatcatcacaacgtcgtcactccttgactcgtcagcgttcctcacaacgtctgattcgtaccatctccatcgagtctgatgtgtccaacatcactgacgatgacgacttgcgtgctgtgaatgagggtgttgctggtgtgcaactggacgtctctgagactgccaacaagggtccacgtcgtgcatctgccactgatgtcactgattccttgggttcaacttcatctgagtacatcgagattccattcgtgaaggagaccttggatgcatccttgccatccgactacctgaagcaggacatcttgaatctgattcagtccctgaaaatctccaagtggtacaacaacaagaagatccaacctgtggcacaagacatgaacctggtcaaaatctctggtgcaatgaccaacgcaatcttcaaggttgagtaccctaagctgccatccttgctgttgcgtatctacggtcctaacatcgacaacatcattgaccgtgagtatgagttgcagatcctggcacgtctgtccttgaagaacatcggtccatcactgtatggctgcttcgtcaacggtcgtttcgagcagttcctggagaactccaagaccttgaccaaggacgacatccgtaactggaagaactctcaacgtatcgcacgtcgtatgaaggagttgcatgttggtgttccactgttgtcatctgaacgtaagaacggttctgcctgttggcagaagatcaaccagtggttgcgtaccatcgagaaggttgaccagtgggttggtgatcctaagaacattgagaactccttgttgtgtgagaactggtccaagttcatggacattgtggatcgttaccacaagtggctgatctcccaagaacagggtatcgagcaagtgaacaagaatctgatcttctgccacaatgatgcacagtatggcaacttgctgttcactgcacctgtcatgaacactccatcactgtacactgcaccatcctccacctcactgacctcacagtcatcatccttgttcccatcatcatccaacgtgattgttgatgacatcatcaacccacctaagcaggagcaatcacaagactccaagttggtggtgatcgacttcgagtatgctggtgccaaccctgctgcctatgaccttgccaatcacctgtctgagtggatgtatgactacaacaacgccaaggcaccacatcagtgccacgctgatcgttaccctgacaaggaacaggtcttgaacttcttgtactcctatgtctcacatcttcgtggtggtgctaaggaacctatcgatgaagaggttcaacgtctgtacaagtccatcattcagtggcgtccaactgttcaactcttctggtcactgtgggcaatccttcagtctggcaagttggagaagaaggaagcatccactgcaatcactcgtgaagagatcggtcctaacggcaagaagtacatcatcaagactgaacctgagtcacctgaagaagacttcgttgagaacgacgatgaacctgaagctggtgtctccatcgacaccttcgactacatggcatacggtcgtgacaagatcgctgtcttctggggtgatctgattggccttggcatcatcactgaagaagagtgcaagaacttctcctccttcaagttcctcgacacttcctacttgtaa

SEQ ID NO: 8 - DNA, 919 bp, synthetic construct
  source 1..919: mol_type = other DNA; note = Nucleotide sequence of AtIPKS gene
gaacgagctggagaagattcacgatgagaatctggaggttgttgcctgtcagcttcgtcaagctatgctggagggttctgcaccttccaaggtcatcggtatggactggtccaagcgtcctggttcatctgaaatctcttgtgatgtggatgacatcggtgatcagaagtcctctgagttctccaagttcgttgttgtccacggtgctggttccttcggtcacttccaggcatcacgttctggtgttcacaagggtggtcttgagaagcctatcgtgaaggctggcttcgttgctactcgtatctctgtcaccaacctgaaccttgagatcgttcgtgcacttgcacgtgagggtatccctaccatcggtatgtctccattctcatgtggttggtcaacctccaagcgtgatgttgcatctgctgatcttgccactgttgccaagaccatcgactctggcttcgttcctgttctgcatggtgatgctgtcctggacaacatccttggctgcaccatcttgtctggtgatgtgatcatccgtcatcttgctgatcacttgaagcctgagtatgttgtgttcctgactgatgtccttggtgtgtacgatcgtccaccatcaccatctgaaccagatgctgtgctgctgaaggagatcgctgttggtgaagatggttcatggaaggttgtcaacccactgttggagcacactgacaagaaggttgactactctgttgcagcacacgacactactggtggtatggagaccaaaatctctgaagctgccatgattgctaagctgggtgtggatgtgtacatcgtcaaggctgccaccactcactcacagcgtgcactgaacggtgacttgcgtgactctgttcctgaagactggcttggtactatcatccgtttctccaagtaa

SEQ ID NO: 9 - DNA, 19 bp, synthetic construct
  source 1..19: mol_type = other DNA; note = T7 promoter sequence
taatacgactcactatagg

SEQ ID NO: 10 - DNA, 47 bp, synthetic construct
  source 1..47: mol_type = other DNA; note = T7 terminator sequence
tagcataaccccttggggcctctaaacgggtcttgaggggttttttg

SEQ ID NO: 11 - DNA, 40 bp, synthetic construct
  source 1..40: mol_type = other DNA; note = P1 of T7 tdiAS
gaatcctaatacgactcactataggatggcaccatctaag

SEQ ID NO: 12 - DNA, 47 bp, synthetic construct
  source 1..47: mol_type = other DNA; note = P2 of T7 tdiAS
gtattcagtagccatcctatagtgagtcgtattacaaaaaacccctc

SEQ ID NO: 13 - DNA, 49 bp, synthetic construct
  source 1..49: mol_type = other DNA; note = P3 of T7 tdiBS
ttgaggggttttttgtaatacgactcactataggatggctactgaatac

SEQ ID NO: 14 - DNA, 47 bp, synthetic construct
  source 1..47: mol_type = other DNA; note = P4 of T7 tdiBS
aagagctgcgtgcatcctatagtgagtcgtattacaaaaaacccctc

SEQ ID NO: 15 - DNA, 49 bp, synthetic construct
  source 1..49: mol_type = other DNA; note = P5 of T7 tdiCS
ttgaggggttttttgtaatacgactcactataggatgcacgcagctctt

SEQ ID NO: 16 - DNA, 47 bp, synthetic construct
  source 1..47: mol_type = other DNA; note = P6 of T7 tdiCS
accaatagaacccatcctatagtgagtcgtattacaaaaaacccctc

SEQ ID NO: 17 - DNA, 49 bp, synthetic construct
  source 1..49: mol_type = other DNA; note = P7 of T7 tdiDS
ttgaggggttttttgtaatacgactcactataggatgggttctattggt

SEQ ID NO: 18 - DNA, 48 bp, synthetic construct
  source 1..48: mol_type = other DNA; note = P8 of T7 tdiDS
atggtctgttaaccatcctatagtgagtcgtattacaaaaaacccctc

SEQ ID NO: 19 - DNA, 50 bp, synthetic construct
  source 1..50: mol_type = other DNA; note = P9 of T7 tdiES
ttgaggggttttttgtaatacgactcactataggatggttaacagaccat

SEQ ID NO: 20 - DNA, 47 bp, synthetic construct
  source 1..47: mol_type = other DNA; note = P10 of T7 tdiES
accgtagattttcatcctatagtgagtcgtattacaaaaaacccctc

SEQ ID NO: 21 - DNA, 49 bp, synthetic construct
  source 1..49: mol_type = other DNA; note = P11 of T7 spfS
ttgaggggttttttgtaatacgactcactataggatgaaaatctacggt

SEQ ID NO: 22 - DNA, 35 bp, synthetic construct
  source 1..35: mol_type = other DNA; note = P12 of T7 spfS
aagcttcaaaaaacccctcaagacccgtttagagg

SEQ ID NO: 23 - DNA, 38 bp, synthetic construct
  source 1..38: mol_type = other DNA; note = P13 of T7 ScCKS
gaatcctaatacgactcactataggatggttcaagagt

SEQ ID NO: 24 - DNA, 48 bp, synthetic construct
  source 1..48: mol_type = other DNA; note = P14 of T7 ScCKS
cttctccagctcgttccctatagtgagtcgtattacaaaaaacccctc

SEQ ID NO: 25 - DNA, 50 bp, synthetic construct
  source 1..50: mol_type = other DNA; note = P15 of T7 AtIPKS
ttgaggggttttttgtaatacgactcactatagggaacgagctggagaag

SEQ ID NO: 26 - DNA, 35 bp, synthetic construct; identical to SEQ ID NO: 22
  source 1..35: mol_type = other DNA; note = P16 of T7 AtIPKS

SEQ ID NO: 27 - DNA, 21 bp, synthetic construct
  source 1..21: mol_type = other DNA; note = Primer tdiA-F
tccgtcaagtgcatggatgtc

SEQ ID NO: 28 - DNA, 21 bp, synthetic construct
  source 1..21: mol_type = other DNA; note = Primer tdiA-R
cagaccacgctcacgcaggac

SEQ ID NO: 29 - DNA, 21 bp, synthetic construct
  source 1..21: mol_type = other DNA; note = Primer tdiB-F
gcactgaagaagctgggtaac

SEQ ID NO: 30 - DNA, 21 bp, synthetic construct
  source 1..21: mol_type = other DNA; note = Primer tdiB-R
acggaaaccgaagtcaccagc

SEQ ID NO: 31 - DNA, 21 bp, synthetic construct
  source 1..21: mol_type = other DNA; note = Primer tdiC-F
atctctcgtaagccaatctgc

SEQ ID NO: 32 - DNA, 21 bp, synthetic construct
  source 1..21: mol_type = other DNA; note = Primer tdiC-R
gacgatgacgacacgggaacc

SEQ ID NO: 33 - DNA, 21 bp, synthetic construct
  source 1..21: mol_type = other DNA; note = Primer tdiD-F
atgttcgtctggctggaactc

SEQ ID NO: 34 - DNA, 21 bp, synthetic construct
  source 1..21: mol_type = other DNA; note = Primer tdiD-R
gcaacgatcgaccagaccagc

SEQ ID NO: 35 - DNA, 21 bp, synthetic construct
  source 1..21: mol_type = other DNA; note = Primer tdiE-F
aagaccttgggtttgtggaac

SEQ ID NO: 36 - DNA, 21 bp, synthetic construct
  source 1..21: mol_type = other DNA; note = Primer tdiE-R
gacgtcggaaccaggtgcagc

SEQ ID NO: 37 - DNA, 21 bp, synthetic construct
  source 1..21: mol_type = other DNA; note = Primer sfp-F
tctcactctggacgttgggtg

SEQ ID NO: 38 - DNA, 21 bp, synthetic construct
  source 1..21: mol_type = other DNA; note = Primer sfp-R
tgcagatgcgatgagacgttg

SEQ ID NO: 39 - DNA, 21 bp, synthetic construct
  source 1..21: mol_type = other DNA; note = Primer ScCK-F
ggtcctaacggcaagaagtac

SEQ ID NO: 40 - DNA, 21 bp, synthetic construct
  source 1..21: mol_type = other DNA; note = Primer ScCK-R
ggaggagaagttcttgcactc

SEQ ID NO: 41 - DNA, 21 bp, synthetic construct
  source 1..21: mol_type = other DNA; note = Primer AtIPK-F
ccactgttggagcacactgac

SEQ ID NO: 42 - DNA, 21 bp, synthetic construct
  source 1..21: mol_type = other DNA; note = Primer AtIPK-R
ggagaaacggatgatagtacc